(19) United States
(12) Patent Application Publication
(10) Pub. No.: US 2013/0053668 A1
(43) Pub. Date: Feb. 28, 2013

(54) KIT AND METHOD FOR DETECTING BLOOD SUGAR

(71) Applicant: Tsu-Tai Lin, Taipei City (TW)

(72) Inventor: Tsu-Tai Lin, Taipei City (TW)

(73) Assignee: COMPOSE ELEMENT LIMITED, Taipei City (TW)

(21) Appl. No.: 13/594,420

(22) Filed: Aug. 24, 2012

Related U.S. Application Data
(60) Provisional application No. 61/527,675, filed on Aug. 26, 2011.

(51) Int. Cl. A61B 5/145

(52) U.S. Cl. 600/362

(57) ABSTRACT

The present invention relates to a kit for detecting blood sugar. The present invention also relates to a method for detecting blood sugar according to the kit. The kit comprises an adhesive sweat absorption device comprising: a sweat absorption patch for absorbing sweat of a subject; a detector layer for generating electronic signals according to the sugar level of sweat absorbed by the sweat absorption patch; a signal amplifier for amplifying electronic signals generated by the detector layer; a wireless transmitter for sending amplified electronic signals generated by the signal amplifier to a remote signal receiver, in order to calculate blood sugar level; and a power supply device for supplying electric power required for operation of detector layer.

### FIELD OF THE INVENTION

This invention relates to detection of blood sugar, especially to a kit and a method for detecting blood sugar from sweat.

### BACKGROUND OF THE INVENTION

Generally, there is a need for patients with diabetes to detect blood sugar level on their own. Result of blood sugar level can be immediately obtained via detection for diet control and blood sugar level maintenance. Moreover, clinicians can provide appropriate treatment for patients according to the daily records. In the past, urine test strips were applied to detect blood sugar level based on colorimetric method. Nowadays, most of the detections rely on finger lancing. The blood specimen on the test strip is read by the blood sugar meter and the blood sugar level is then obtained from calculation of the blood sugar meter. However, it is usual that patients with diabetes monitor their blood sugar intensively within a day. The pain and infection risks of patients for measuring are increased via puncture treatment.

In order to solve the above shortcomings, the inventor of the present application makes efforts on researches, and finally submits a well-designed and effective invention.

### BRIEF DESCRIPTION OF THE DRAWINGS

FIG. 1 is a schematic diagram of a sweat absorption patch for use in a kit of detecting blood sugar of the invention.

FIG. 2 is a schematic diagram showing a preferred embodiment of a kit of detecting blood sugar of the invention.

FIG. 3 is a schematic diagram of an embodiment of a GOD or Gluc-DOR assay according to the invention.

### SUMMARY OF THE INVENTION

The present invention relates to a kit for detecting blood sugar comprising a sweat absorption patch for absorbing sweat of a subject; a detector layer for generating electronic signals according to the sugar level of sweat absorbed by the sweat absorption patch; a signal amplifier for amplifying electronic signals generated by the detector layer; a wireless transmitter for sending amplified electronic signals generated by the signal amplifier to a remote signal receiver to calculate blood sugar level; and a power supply device for supplying electric power required for operation thereof.

The present invention also relates to a method for detecting blood sugar using the kit for detecting the blood sugar of the present invention, comprising: adhering the sweat absorption patch on a subject to be detected for absorbing sweat; generating electronic signals based on the sugar level of the absorbed sweat from the detector layer; amplifying the electronic signals generated from the detector layer through the signal amplifier; transmitting the amplified electronic signals from the signal amplifier to the remote signal receiver through the wireless transmitter; and calculating blood sugar level from the electronic signals received by the remote signal receiver.

### DETAILED DESCRIPTION OF THE INVENTION

An objective of the present invention is to provide a kit for detecting blood sugar from sweat, comprising a sweat absorption patch, a test stripe, and a sugar meter with built-in sweat sugar and blood sugar conversion program.

Another objective of the present invention is to provide a method for detecting blood sugar from sweat, which is measured by absorbing sweat of a subject to be detected so as to detect the blood sugar level of the subject. The pain and infection risks of patients with puncture treatment can be avoided.

The articles “a” and “an” are used herein to refer to one or to more than one (i.e., to at least one) of the grammatical object of the article. By way of example, “an element” means one element or more than one element.

Throughout the claims, the term “or” is employed to describe “and/or”.

Throughout the description and claims of this specification, the words “comprise”, “have”, “include”, “contain” and variations of the words, for example “comprising” and “comprises”, means “including but not limited to”, and is not intended to (and does not) exclude other moieties, additives, components, integers or steps.

Therefore, the present invention provides a kit for detecting blood sugar comprising: a sweat absorption patch, comprising a polyurethane membrane layer, a pressure sensitive adhesion layer, a meltdown nonwoven and a hydrogel layer, for absorbing sweat of a subject to be detected via the hydrogel layer; a detector layer, placed above the hydrogel layer, for detecting a sugar level of the sweat absorbed by the sweat absorption patch and generating corresponding electronic signals; a signal amplifier, coupled to the detector layer, for amplifying the electronic signals generated by the detector layer; a wireless transmitter, coupled to the signals amplifier, for sending the amplified electronic signals generated by the signal amplifier to a remote signal receiver, building internally a conversion function between the sugar level of the sweat and a blood sugar level, to calculate the blood sugar level of the subject to be detected; and

a power supply device, placed above the polyurethane membrane of the sweat absorption patch and respectively connected to the detector layer, the signal amplifier and the wireless transmitter, for supplying electric power required for operation thereof.

The present invention also provides a method for detecting blood sugar using the kit of the present invention, comprising:
a. adhering the sweat absorption patch on a subject to be detected for absorbing sweat;
b. detecting a sugar level of the absorbed sweat via the detector layer located on an upper side of the hydrogel layer in the sweat absorption patch of the kit and generating corresponding electronic signals;
c. amplifying the electronic signals generated by the detector layer through the signal amplifier;
d. transmitting the amplified electronic signals from the signal amplifier to the remote signal receiver through the wireless transmitter; and
e. calculating blood sugar level of the subject to be detected from the electronic signals received by the remote signal receiver, using a conversion function between the sugar level of the sweat and the blood sugar level.

In a preferred embodiment, the kit further comprises a remote signal receiver for receiving electronic signals sent by the wireless transmitter and calculating blood sugar level from the electronic signals. More preferably, the remote signal receiver has a display unit to display value of the sweat sugar and/or the blood sugar.

In a preferred embodiment, interface of the wireless transmitter is radio, WLAN, infrared ray, bluetooth, radio frequency, GSM, PHS, CDMA or other wireless interfaces.

In a preferred embodiment, the power supply device is an electric power storage device

In a preferred embodiment, the sweat absorption patch comprises: a polyurethane membrane layer (backbone material layer) which is a an unidirectional penetrating membrane with tension, waterproof, and water vapor permeability; a pressure sensitive adhesion layer which is of a hydrophobic material coated on the polyurethane membrane layer to fit with skin; a meltblown nonwoven which is of a hydrophobic material with multi-directional elastic tension; and a hydrogel layer which is of a hydrophilic material. The meltblown nonwoven and the hydrogel layer are adhered together by UV irradiation to form an interpenetrating polymer network, and part of fibers of the meltblown nonwoven are exposed and stably adhered to the pressure sensitive adhesive layer resulting in multi-directional elasticity of the hydrogel layer to be adapted to different parts of the skin. Meanwhile, the detector layer may be interposed between the hydrogel layer and the meltdown nonwoven for adhering together by the UV irradiation or may be placed on the meltdown nonwoven after the UV irradiation.

More preferably, the hydrogel layer comprises a monomer, a plasticizer, a photoinitiator, a cross-linking agent and a thickener.

In a preferred embodiment, weight ratios of component in the hydrogel layer are:

| | | |
|---|---|---|
| acrylic amide monomer | 15 to 30 | units; |
| acrylic sulfonate monomer | 10 to 50 | units; |
| glycerol | 15 to 45 | units; |
| photoinitiator | 0.01 to 0.1 | units; and |
| bifunctional ester monomer | 0.01 to 0.2 | units. |
| with unsaturated double bond | | |

In a preferred embodiment, the detector layer comprises reduction electrodes for detecting potential difference in an oxidation reaction.

### EXAMPLES

The examples below are non-limiting and are merely representative of various aspects and features of the present invention.

### Example 1

### Kit for Detecting Blood Sugar

### Adhesive Sweat Absorption Device

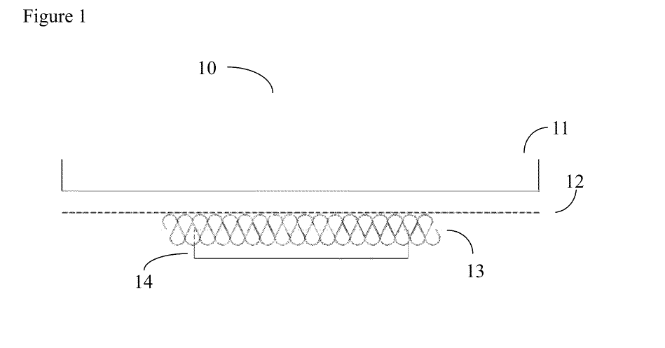

A sweat absorption patch 10 used in a kit of detecting blood sugar according to the present invention is shown in FIG. 1. The sweat absorption patch 10 comprises: (a) a polyurethane membrane layer 11 which is an unidirectional penetrating membrane with tension, waterproof, and water vapor permeability; (b) a pressure sensitive adhesion layer 12 which is of a hydrophobic material coated on the polyurethane membrane layer 11 to fit with skin; (c) a meltblown nonwoven 13 which is of a hydrophobic material with multi-directional elastic tension; and (d) a hydrogel layer 14 which is of a hydrophilic material. The meltblown nonwoven 13 and the hydrogel layer 14 were adhered together by UV irradiation to form an interpenetrating polymer network, and part of fibers of the meltblown nonwoven 13 were exposed and stably adhered to the pressure sensitive adhesive layer resulting in multi-directional elasticity of the hydrogel layer 14 to be adapted to different parts of skin.

The polyurethane membrane layer 11 in the patch 10 was a an unidirectional penetrating membrane with tension, waterproof, and water vapor permeability, providing thermoplastic deformable elasticity and tension for satisfying the requirement of covering with various skin angles. The polyurethane membrane layer 11 also provided the effect of waterproof, bacteria-resisting, ventilation and heat dissipation.

The pressure sensitive adhesion layer 12 of the patch was a hydrophobic material coated on the polyurethane membrane layer 11 to fit with skin.

The hydrogel layer 14 of the patch had characteristics of water absorption and that can be adhered onto the skin for a long time to absorb sweat and the harm on epidermal cells and uncomfortable feeling when removing the patch from the skin.

The hydrogel layer 14 of the patch was prepared as follows:
(a) providing a mixture prepared by steps comprising:
   (I) stirring and mixing photoinitiator and acrylic amide monomer until dissolved;
   (II) adding glycerol and mixing until dissolved;
   (III) adding acrylic sulfonate monomer and mixing until dissolved; and
   (IV) further adding glycerol and stirring for mix;
(b) further providing a mixture prepared by steps comprising stirring and mixing photoinitiator and bifunctional ester monomer with unsaturated double bond;
(c) stirring and mixing the mixture from step (a) and (b);
(d) applying UV radiation on the mixture of step (c) for cross-linking polymerization to obtain the hydrogel layer 14.

The weight ratios of the components described above were:

| | | |
|---|---|---|
| acrylic amide monomer | 15 to 30 | units; |
| acrylic sulfonate monomer | 10 to 50 | units; |
| glycerol | 15 to 45 | units; |
| photoinitiator | 0.01 to 0.1 | units; and |
| bifunctional ester monomer | 0.01 to 0.2 | units. |
| with unsaturated double bond | | |

The adhesive sweat absorption device used in the kit still comprises a detector layer 20 which generated electronic signals according to the sugar level of sweat absorbed by the sweat absorption patch 10.

The adhesive sweat absorption device used in the kit still comprises a signal amplifier 30 which amplified electronic signals generated by the detector layer 20.

The adhesive sweat absorption device used in the kit still comprises a wireless transmitter 40 which sent amplified electronic signals generated by the signal amplifier 30 to a remote signal receiver 60 to calculate blood sugar level.

The adhesive sweat absorption device used in the kit still comprises a power supply device 50 which supplied electric power required for operation of detector layer 20.

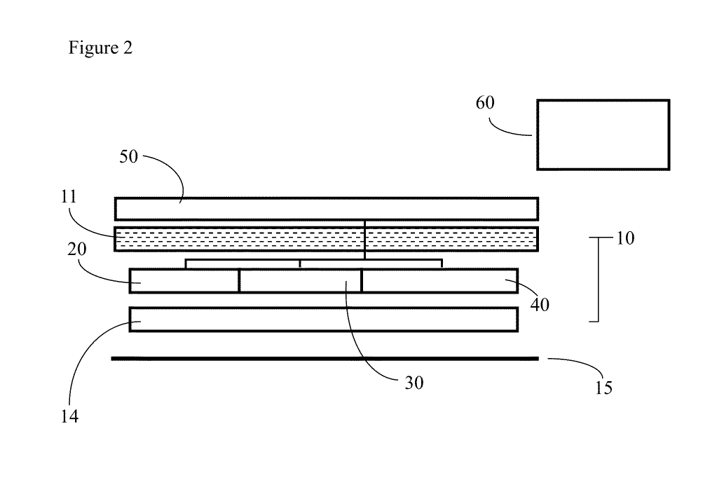

A preferred embodiment of a kit for detecting blood sugar according to the present invention is shown as FIG. 2. The detector layer 20 (or sensor) was located on the hydrogel layer 14 and covered with a backbone material layer. The backbone material layer could be a PU film or an adhesive layer. A battery layer located on the backbone material layer was to provide electric power required for detector layer 20 (or sensor). The lower side of the hydrogel layer 14 has a releasing layer 15, serving as a protection envelope when the sweat absorption patch 10 is not in use.

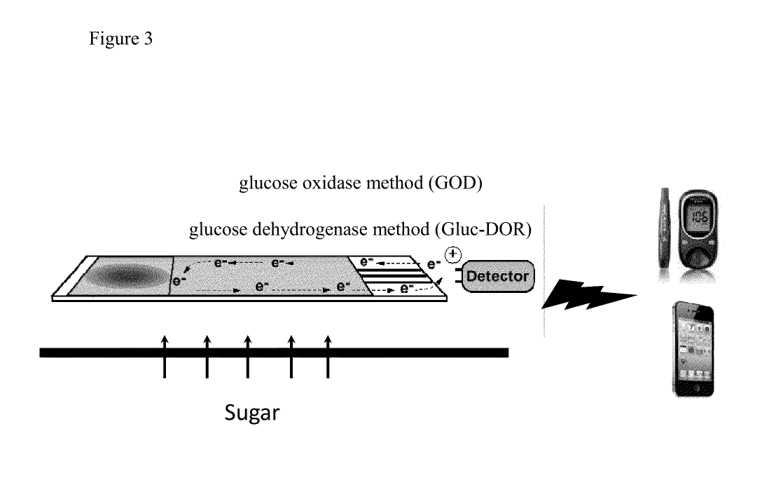

A GOD or Gluc-DOR assay according to the present invention is shown as FIG. 3. The detector layer 20 comprises reduction electrodes for detecting potential difference in an oxidation reaction. The sweat was absorbed through the hydrogel layer 14 and detected by detector layer 20. The sugar level of sweat was detected by electrochemical methods such as glucose dehydrogenase(Gluc-GOD) assay or glucose oxidase(GOD) assay. After signal processing, the resulting electronic signals were transmitted to sugar meter or smart phone to display the value of sweat sugar and/or blood sugar.

The kit further included a remote signal receiver 60 for receiving electronic signals sent by the wireless transmitter 40 and calculating blood sugar level from the electronic signals. The method of calculation was performed through a built-in sweat sugar level and blood sugar level conversion function of the remote signal receiver 60. The remote signal receiver 60 had a display unit to display the value of blood sugar level.

### Example 2

### Building the Sweat Sugar Level and Blood Sugar Level Conversion Function

First, the kit of the present invention detected sweat sugar of a subject and used the remote signal receiver 60 to read and display the value of sweat sugar.

Then, commercial photochemical blood sugar meter or electrochemical blood sugar meter was used to detect blood sugar of the same subject at the same time, and the value of blood sugar was displayed on blood sugar meter.

The values of sweat sugar and blood sugar were recorded. The value of sweat sugar was set as an independent variable X, and the value of blood sugar was set as a dependent variable Y. At least 100 samples were processed by simple linear regression analysis to construct a conversion function of X and Y. Any of the value of sweat sugar can be directly calculated into a corresponding value of blood sugar using the conversion function.

One skilled in the art readily appreciates that the present invention is well adapted to carry out the objects and obtain the ends and advantages mentioned, as well as those inherent therein. The kits, processes, and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

While the invention has been described and exemplified in sufficient detail for those skilled in this art to make and use it, various alternatives, modifications, and improvements should be apparent without departing from the spirit and scope of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A kit for detecting blood sugar, comprising:
a sweat absorption patch, comprising a polyurethane membrane layer, a pressure sensitive adhesion layer, a meltdown nonwoven and a hydrogel layer, for absorbing sweat of a subject to be detected via the hydrogel layer;
a detector layer, placed above the hydrogel layer, for detecting a sugar level of the sweat absorbed by the sweat absorption patch and generating corresponding electronic signals;
a signal amplifier, coupled to the detector layer, for amplifying the electronic signals generated by the detector layer;
a wireless transmitter, coupled to the signals amplifier, for sending the amplified electronic signals generated by the signal amplifier to a remote signal receiver, building internally a conversion function between the sugar level of the sweat and a blood sugar level, to calculate the blood sugar level of the subject to be detected; and
a power supply device, placed above the polyurethane membrane of the sweat absorption patch and respectively connected to the detector layer, the signal amplifier and the wireless transmitter, for supplying electric power required for operation thereof.

2. The kit of claim 1, wherein the sweat absorption patch further comprises a releasing layer, located at a lower side of the hydrogel layer, serving as a protection envelope when the sweat absorption patch is not in use.

3. The kit of claim 1, wherein the remote signal receiver comprises a display unit to display value of the sweat sugar and/or the blood sugar.

4. The kit of claim 1, wherein interface of the wireless transmitter is radio, WLAN, infrared ray, bluetooth, radio frequency, GSM, PHS, CDMA or other wireless interfaces.

5. The kit of claim 1, wherein the power supply device is an electric power storage device.

6. The kit of claim 1, wherein the sweat absorption patch comprises:
a. the polyurethane membrane layer which is an unidirectional penetrating membrane with tension, waterproof, and water vapor permeability;
b. the pressure sensitive adhesion layer which is of a hydrophobic material coated on the polyurethane membrane layer to fit with skin;
c. the meltblown nonwoven which is of a hydrophobic material with multi-directional elastic tension; and
d. the hydrogel layer which is of a hydrophilic material,
wherein the meltblown nonwoven and the hydrogel layer are adhered together by UV irradiation to form an interpenetrating polymer network, and part of fibers of the meltblown nonwoven are exposed and stably adhered to the pressure sensitive adhesive layer resulting in multi-directional elasticity of the hydrogel layer to be adapted to different parts of the skin, and wherein the detector layer is interposed between the hydrogel layer and the meltdown nonwoven for adhering together by the UV irradiation.

7. The kit of claim 6, wherein the hydrogel layer comprises a monomer, a plasticizer, a photoinitiator, a cross-linking agent and a thickener.

8. The kit of claim 6, wherein weight ratios of component in the hydrogel layer are:

| | | |
|---|---|---|
| acrylic amide monomer | 15 to 30 | units; |
| acrylic sulfonate monomer | 10 to 50 | units; |
| glycerol | 15 to 45 | units; |
| photoinitiator | 0.01 to 0.1 | units; and |
| bifunctional ester monomer | 0.01 to 0.2 | units. |
| with unsaturated double bond | | |

9. The kit of claim 1, wherein the detector layer comprises reduction electrodes for detecting potential difference in oxidation.

10. A method for detecting blood sugar using the kit for detecting blood sugar of claim 1, comprising:
a. adhering the sweat absorption patch on a subject to be detected for absorbing sweat;
b. detecting a sugar level of the absorbed sweat via the detector layer located on an upper side of the hydrogel layer in the sweat absorption patch of the kit and generating corresponding electronic signals;
c. amplifying the electronic signals generated by the detector layer through the signal amplifier;
d. transmitting the amplified electronic signals from the signal amplifier to the remote signal receiver through the wireless transmitter; and
e. calculating blood sugar level of the subject to be detected from the electronic signals received by the remote signal receiver, using a conversion function between the sugar level of the sweat and the blood sugar level.
